(19) United States
(12) Patent Application Publication
(10) Pub. No.: US 2022/0087834 A1
(43) Pub. Date: Mar. 24, 2022

(54) PROSTHETIC FOOT COMPONENT

(71) Applicant: Ottobock SE & Co. KGAA, Duderstadt (DE)

(72) Inventor: Martin PUSCH, Duderstadt (DE)

(73) Assignee: Ottobock SE & Co. KGAA, Duderstadt (DE)

(21) Appl. No.: 17/424,858

(22) PCT Filed: Jan. 24, 2020
(86) PCT No.: PCT/EP2020/051780

§ 371 Date: Jul. 21, 2021

(30) Foreign Application Priority Data
Jan. 25, 2019 (DE) 10 2019 101 843.9

(51) Int. Cl. A61F 2/66

(52) U.S. Cl. CPC A61F 2/66; A61F 2002/5001

(57) ABSTRACT

The invention relates to a prosthetic foot component comprising: a. a proximal fastening system for securing the prosthetic foot component to a proximal component or to a patient; b. a retaining portion situated distally relative to the fastening system and connected to the latter; and c. a main spring extending in a front-foot region and coupled to the retaining portion, wherein: said retaining portion is mounted on the main spring to tilt on the sagittal plane; and a posterior limiting element, which limits the displacement of the retaining portion away from the main spring, is provided between the main spring and the retaining portion.

The invention relates to a prosthetic foot insert with a proximal fastening device for securing the prosthetic foot insert to a proximal component or to a patient, a holder which is arranged distally with respect to the fastening device and is connected to the fastening device, and a main spring which extends into a forefoot region and is coupled to the holder.

Prosthetic foot inserts are part of a prosthetic fixture, for example for below-knee amputees. Prosthetic foot inserts can be provided with a prosthesis covering, which can be made of a plastic, in order to achieve the most natural possible appearance and to provide further functionality. The prosthetic foot inserts can be fastened to an ankle joint or, without a joint, to a lower-leg tube or a lower-leg socket. The fastening device is generally designed as what is called a pyramid adapter, via which multiple settings and orientations of the prosthetic foot insert can be adjusted and fixed in relation to the proximal component, i.e. to the lower-leg tube, the prosthesis socket or the ankle joint. The fastening device is fastened to a holder, on which in turn a spring, extending in the forefoot direction, for example a forefoot spring or a roof spring, can be arranged. In order to damp an impact at heel strike, an elastic heel element is provided, which is fastened to the holder, if appropriate with interpositioning of intermediate pieces. Examples of a prosthetic insert are described in EP 2 420 212 A1, EP 1 976 463 A1, US 2005/0038525 A1 or EP 2 688 522 B1.

The problems with prosthetic foot inserts from the prior art are the installation space that may be required, an unsatisfactory bending behavior, an uneven rollover behavior, and difficulties in compensating for unevenness. In addition, complex shapes are sometimes necessary, which increase the production costs and cause difficulties in the optimal utilization of materials.

The object of the present invention is therefore to make available a prosthetic foot insert which permits optimized behavior while standing and also while walking, in particular by providing sufficient stability while walking, without compromising comfort during walking.

According to the invention, this object is achieved by a prosthetic foot insert having the features of the main claim. Advantageous embodiments and developments of the invention are disclosed in the subclaims, the description and the figures.

In the prosthetic foot insert with a proximal fastening device for securing the prosthetic foot insert to a proximal component of a prosthesis or to a patient, a holder arranged distally with respect to the fastening device and coupled to the fastening device, and a main spring which extends into a forefoot region and is coupled to the holder, provision is made that the holder is mounted on the main spring so as to be tiltable in the sagittal plane, wherein a posterior limiting element is arranged between the main spring and the holder and limits a displacement of the holder away from the main spring. The prosthetic foot insert is either designed as a separate structural part, which can be secured to a distal prosthesis component, for example to a lower-leg tube or a lower-leg socket, or as a prosthetic foot insert which is produced in one piece with a corresponding fastening device for securing to the patient or the user of the prosthetic foot insert, for example with a device for osseointegrated securing to a patient, but also as an integral constituent part of a lower-leg socket.

The prosthetic foot insert can be used in particular as a basis for other structures such as a mechatronic joint, an ML adapter, an adapter for adjusting the heel heights, a hydraulic joint unit or the like.

A posterior limiting element prevents the holder from being displaced away from the main spring beyond a limit that has been set. The posterior limiting element defines the maximum distance of a posterior end of the holder from the main spring, in particular from a posterior end of the main spring, but the reverse movement still remains possible. On the one hand, a deflection of the prosthetic foot insert in the case of a heel load or a heel strike is not impaired or only negligibly impaired and, on the other hand, sufficient stability is provided in the case of a forefoot load during a rollover movement or when bending forward while standing, such that, in addition to a possible elastic yield when standing, tilting in the sagittal plane is limited in the anterior direction about a tilt axis.

The main spring can be designed as a combined spring having at least one distal spring and/or at least one proximal spring. In the case of two distal springs for example, the proximal distal spring forms a medial spring and the distal distal spring forms a base spring or a ground spring, which preferably extends into the heel region of the prosthetic foot insert. The proximal spring is assigned to the holder or to a guide element for the holder. All of the springs are designed in particular as leaf springs. The leaf springs can have a substantially rectangular cross section and, in the longitudinal extent, can have a uniform thickness or also a changing thickness, in particular a thickness tapering in the anterior direction. The springs can be slotted in the forefoot region in order to permit medial-lateral tilting of the prosthetic foot or to form an opening or notch, for example to be able to accommodate a toe strap of a sandal or the like.

The distal spring and the proximal spring of the main spring are preferably secured to each other at points spaced apart from each other in order to form a free space, so as to permit deflection of the prosthetic foot under a load and to utilize the individual spring properties of the separate springs. The springs are in particular made of a composite material, in particular a fiber-reinforced plastic. The springs can be produced from glass fibers, carbon fibers, aramid fibers, Kevlar fibers, Dyneema fibers or other particularly high-strength fibers embedded in a matrix or from combinations thereof.

In a development of the invention, the distal spring and the proximal spring are oriented relative to each other in a biconcave shape in order to form an enlarged free space in the central region between the two springs. The free space is approximately elliptical and permits deflection over a comparatively long spring travel in the midfoot region or forefoot region. The proximal springs can likewise be shaped and oriented biconcavely in relation to each other. With a biconvex shape between the distal spring and the medial spring, a space that increases in the posterior direction can be formed in the posterior region between the distal spring or base spring and the medial spring, in order to permit deep deflection of the holder in the direction of the base spring or ground spring.

In a development of the invention, provision is made that a guide element is fastened to the main spring in an anterior region or a posterior region and extends in the respectively opposite direction, and the holder is mounted on the main spring so as to be tiltable via this guide element, preferably about a pivot axis orthogonal the sagittal plane. By mounting the holder on the main spring via a guide element, different mounting and movement variants can be set and changed, such that individual adaptation or an increase in the number of variants can be achieved. The holder is then not directly connected and secured to a main spring, for example by direct screwing of a leaf spring on the underside of a holder base, but pivotable about an axis transverse to the walking direction, e.g. via the guide element, a hinge or another intermediate element that permits a relative movement between the main spring and the holder. The guide element can be fastened in an anterior or posterior region of the main spring, wherein the posterior region of the fastening device lies behind the fastening device or a pyramid adapter, in particular posterior to a force application point when standing. The anterior region lies anterior to the fastening device, for example a pyramid adapter, or the position of the resulting ground reaction force vector when standing. From the respective fastening region of the guide element, the latter extends in the respectively opposite direction, that is to say, in the case of fastening in the posterior region, in the anterior direction, and, in the case of fastening in the anterior region, in the posterior direction. The holder is mounted on the main spring so as to be tiltable around the guide element in the sagittal plane, such that a pivoting movement, as well as a vertical movement of the holder relative to the main spring, is made possible by the guide element.

In one development, provision is made that an anterior limiting element is arranged between the guide element and the holder or the main spring, which limiting element, in the case of a heel load, limits a displacement of the anterior end of the holder or a displacement of the main spring away from the guide element. As with the posterior limiting element, the anterior limiting element prevents the holder from moving relative to the main spring during a defined loading phase, in order to influence the deflection behavior and rebound behavior of the prosthetic foot insert. By adjusting the length of the anterior and/or posterior limiting element, a spring tension can be changed and the energy transport behavior of the foot during the different gait phases can be adjusted. Likewise, an adaptation to different usage habits, to different purposes of use for the respective user or for different users, to different or changing physical properties of the respective user and the like, can be made by changing the pretensioning and/or the length in order to limit the displacement of an anterior and/or posterior end of the holder.

The respective limiting element can be tensionally rigid and flexible, in order to ensure a precise setting of the possible displacement and the distances from the holder to the spring or from the spring to the limiting element or guide element. The limiting element is designed, for example, as a strap, cable or cord or also as a sleeve guide with a stop element for limiting the maximum distance in each case.

The pretensioning of the one or more limiting elements is adjustable, in particular to make a noticeable distinction between walking with an impulse load and standing with a somewhat static load. During walking, in particular during heel strike but also during the rollover movement, damping is intended to take place with a corresponding displacement of the components relative to one another. During standing, the user is intended to be given a feeling of security, which is achieved by pretensioning the holder against the resilient elements. The pretensioning is preferably between 5% and 60% of the body weight of the user; in particular the pretensioning is between 5% and 40% of the body weight of the user, particularly preferably between 10% and 25% of the body weight of the user. In the latter case, the pretensioning of the holder by the limiting element or the limiting elements with respect to the spring action of the main spring or of other resilient elements or components would be between 10 kg and 25 kg with a body weight of the user of 100 kg, which corresponds to a force of between approximately 98.1 N and 245.25 N.

The main spring and the guide element are preferably designed as leaf springs, in particular as straight leaf springs, which has the advantage that the production of the spring components is particularly simple. In particular, if the main spring or the individual spring components of the main spring are made from a fiber-reinforced plastic, the main spring as a whole can be made comparatively stiff, such that the durability is increased. A stiff spring configuration of the individual spring components permits a high level of durability of the spring components, but the stiff spring configuration does not lead to a particularly stiff prosthetic foot insert at heel strike, since this can be compensated for by the comparatively large spring travel and a long force transmission. The guide element can likewise be designed as a leaf spring, in particular as a metal leaf spring in the form of a spring tongue. Alternatively, the holder can be mounted on the main spring via a hinge or at least one spacer element, such that the guide element is designed as a spring tongue, hinge or spacer element.

To further adjust the walking behavior and to improve the adaptability of the rollover behavior and to protect the distal spring or the distal portions of the main spring, a forefoot pad and/or a heel pad can be fastened to the main spring, for example glued, screwed or plugged on, or held via the respective limiting element, in particular in the case of a heel pad. The pads are preferably arranged on the base spring or ground spring.

In a development of the invention, provision is made that the holder introduces the forces into the main spring via an anterior force introduction region anterior to the fastening device and a posterior force introduction region posterior to the fastening device. It is thereby possible that the holder is supported on the main spring at two points or regions spaced apart in the longitudinal extent of the main spring, possibly via intermediate elements, intermediate plates or also a damper device, possibly also via an intermediate spring. The support in these force introduction regions permits a reduction in the punctiform load and also a controlled introduction of force under different load scenarios. The force introduction regions preferably lie between two end supports on which the main spring is supported in the distal region, for example between the heel pad and the forefoot pad. As a result, at least a four-point bending takes place when the patient is in the stance phase and the foot is set down, such that the maximum bending moment of the main spring is considerably reduced, since the introduction of force is more widely distributed.

At least one of the force introduction regions can be arranged displaceably or exchangeably on the holder or main spring, such that adjusting the position of the respective force introduction region can change the spring properties and the energy transport properties of the prosthetic foot insert during use. The respective position of the force introduction region is preferably set once to adapt the behavior of the prosthetic foot insert to the respective users and can be set and adjusted before each use. In principle, it is also possible to change the position of the force introduction region by motor. The respective motor or drive can then be adjusted via a control device and a sensor arrangement during walking, in order to permit adaptation to different walking speeds, loads or walking situations.

At least one damping device can be arranged between the holder and the main spring in order to make the renewed contact between holder and main spring soft, after the holder has detached from the main spring in the anterior region or in the posterior region, or in order to damp a pivoting movement. Thus, during walking, it is possible to avoid the occurrence of disruptive force peaks and impulses caused by the contact between holder and main spring, which can make the rollover behavior uneven and uncomfortable for the patient. Likewise, the pivoting behavior of the holder relative to the main spring can be adjusted.

The holder can be mounted on the main spring with an adjustable proximal-distal distance, in order to permit an angle setting and also a change in the engagement behavior and engagement time and thus a change in the energy transfer.

In a development of the invention, provision is made that, in the unloaded state of the prosthetic foot insert, the main spring is elastically pretensioned with respect to the guide element by the limiting element and/or the limiting elements, such that the individual components of the prosthetic foot insert, in the unloaded state, are held together solely by the pretensioning of the main spring relative to the guide element. The limiting elements or the limiting element work between the holder and the main spring. The guide element is arranged between the main spring and the holder and, if only vertical forces occur, i.e. no displacement forces in the horizontal direction, the prosthetic foot insert remains stable without further devices for securing the components to each other. Further securing devices or fastening elements serve only to secure against the occurrence of transverse forces or shear forces. The fastening device can be mounted on the holder in a displaceable and/or articulated manner, in order to be able to adapt to different requirements or patients.

A damper can be arranged between the fastening device and the holder, provided that the fastening device is secured as a separate element on the holder. The damper can allow a slight relative movement between the fastening device and the holder and can reduce load peaks and permit a smooth rollover movement without impulses.

In a development of the invention, provision is made that an exchangeable and/or displaceably mounted contact element is arranged between the proximal spring and the distal spring and/or between the distal springs, via which contact element the respective force introduction point and a coupling time and coupling location between the distal spring and the proximal spring or the distal springs can be fixed, set or adjusted. The exchangeable and/or displaceably mounted contact element makes it easy to change and adapt the deflection behavior and force transmission behavior of the prosthetic foot insert.

The holder can be mounted on the main spring so as to be tiltable in the sagittal plane on a movable surface, such that the holder can roll on the main spring during loading and can permit a displacement relative to the main spring. The holder thus has no fixed point of rotation with respect to the main spring, and instead the point of rotation of the holder shifts during the rollover procedure.

The prosthetic foot insert is in particular also suitable and provided for being adapted to different heel heights or desired inclination angles, such that the user can easily carry out an individual adaptation. This can be done in particular by changing the length of one or more limiting elements. The respective limiting element can be exchanged or lengthened or shortened and then fixed in the desired position, e.g. clamped. The holder can be set in the desired angle position to the ground or to the main spring and fixed, e.g. by exchanging pads, damper devices, intermediate pieces, inserts or spacer elements between the holder and the main spring, in order thereby to obtain an optimized orientation of the holder and of the fastening device. On account of the pivotable bearing either in front of or behind the vertical through the fastening device, the adaptation to the respective heel height can be achieved through alignment and setting in the desired position. The position of the bearing points of axes can be adjustable, in order to obtain an orientation and heel height adaptation. If hydraulic or pneumatic dampers or actuating cylinders are present, the desired position and orientation of the holder can be achieved by opening and closing of valves. The holder is mounted on the main spring with an adjustable proximal-distal distance such that, with a uniform change of the distance, an adaptation can be made to different sole thicknesses of shoes. If the distance is changed differently in front of and behind the fastening device, the inclination of the holder is adapted as desired. For this purpose, the front and rear distance can be individually set by adjustment of actuating cylinders or damper devices or by inserts or the like, as has been described above.

Illustrative embodiments of the invention are explained in more detail below with reference to the accompanying figures, in which:

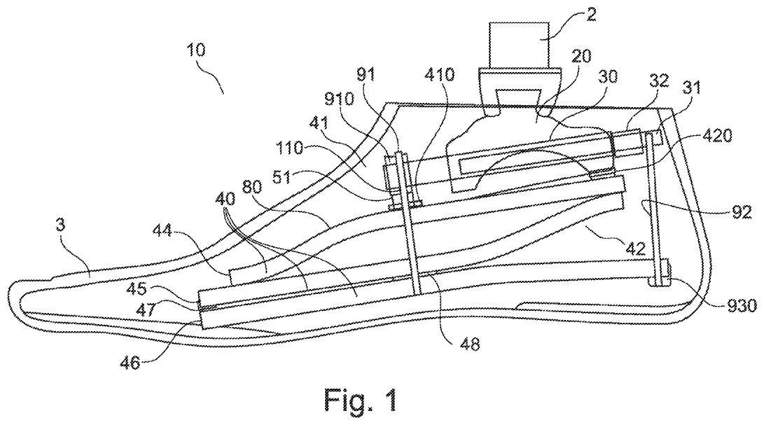

FIG. 1 shows a first embodiment of the prosthetic foot insert in a schematic sectional view;

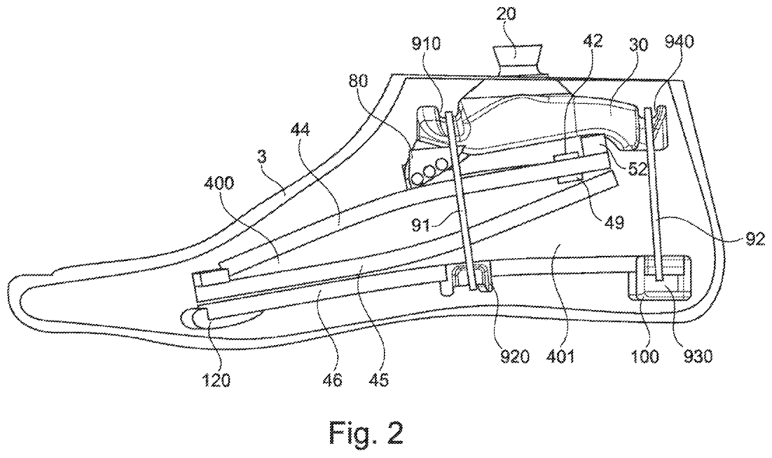

FIG. 2 shows a variant of FIG. 1 with a hinge;

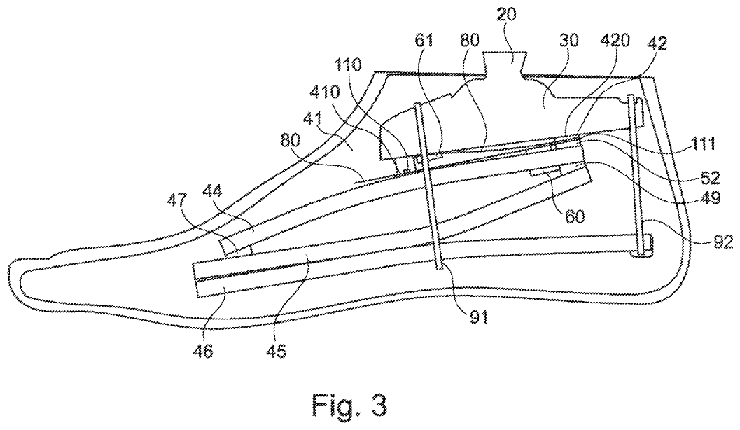

FIG. 3 shows a variant of FIG. 1 with a folded guide element;

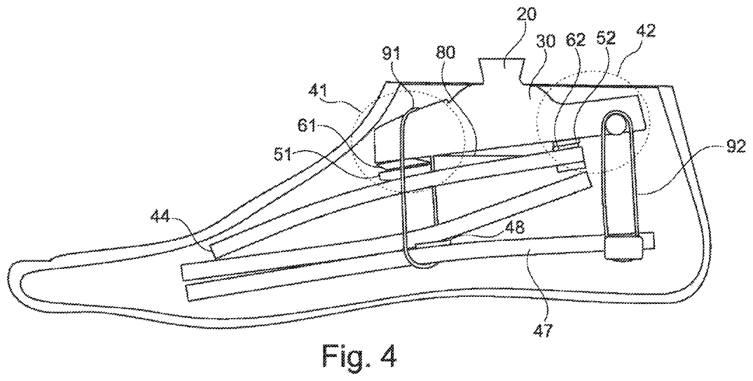

FIG. 4 shows a variant of the invention with a posteriorly protruding holder;

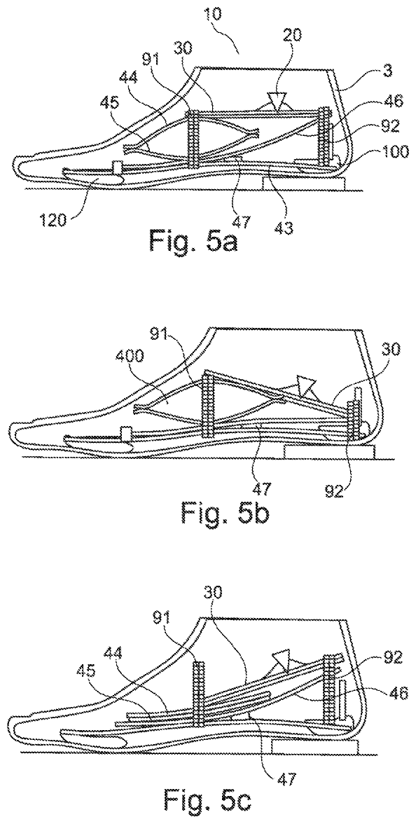

FIGS. 5a-5c show a variant of FIG. 1 with pads in different loading states;

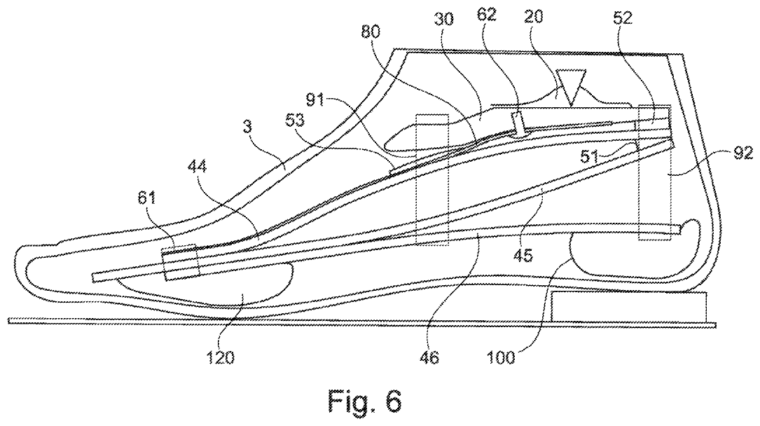

FIG. 6 shows a variant of the invention with a pad between the guide element and the main spring;

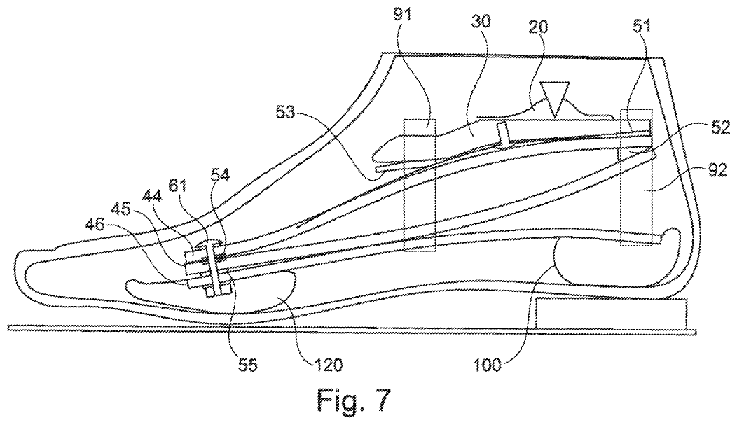

FIG. 7 shows a variant of FIG. 6;

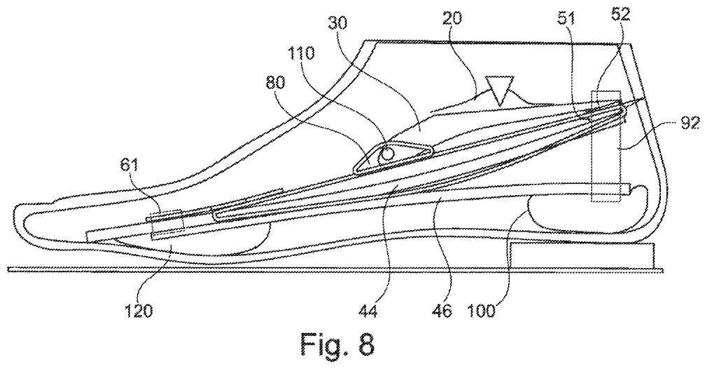

FIG. 8 shows a variant with a hinge and two springs;

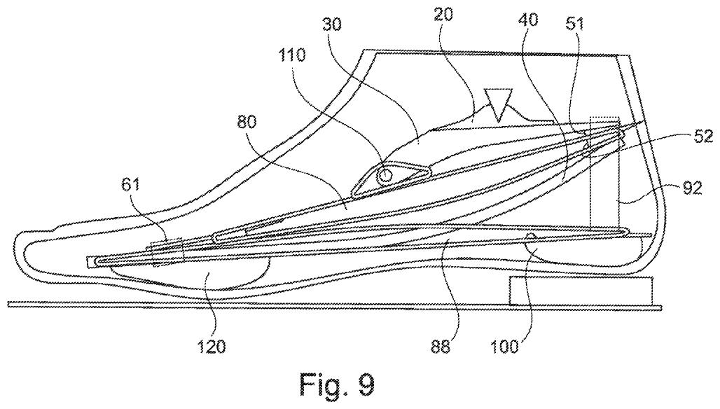

FIG. 9 shows a variant of FIG. 8;

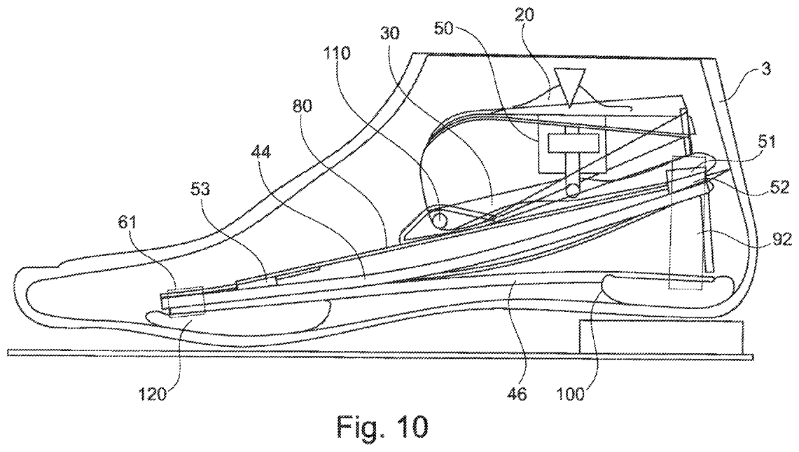

FIG. 10 shows a variant with a height adjustment device in the holder;

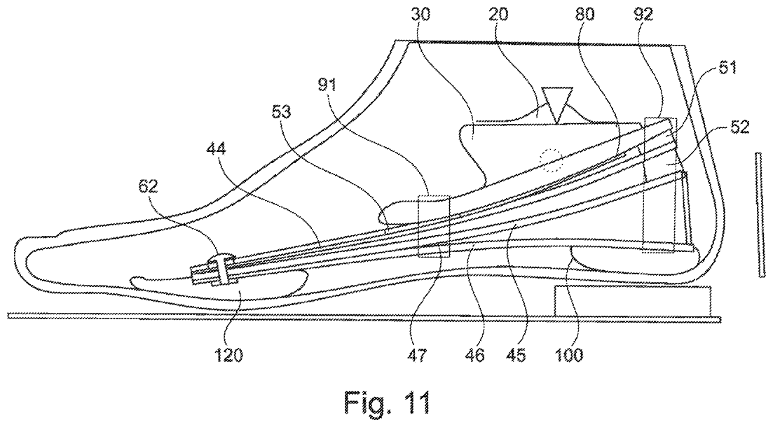

FIG. 11 shows a variant with two substantially parallel springs, and with a base spring curved in the opposite direction;

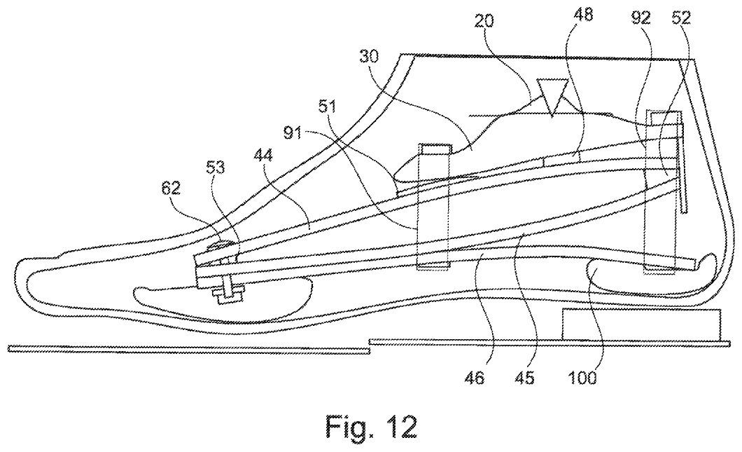

FIG. 12 shows a variant with a displaceable contact between holder and main spring;

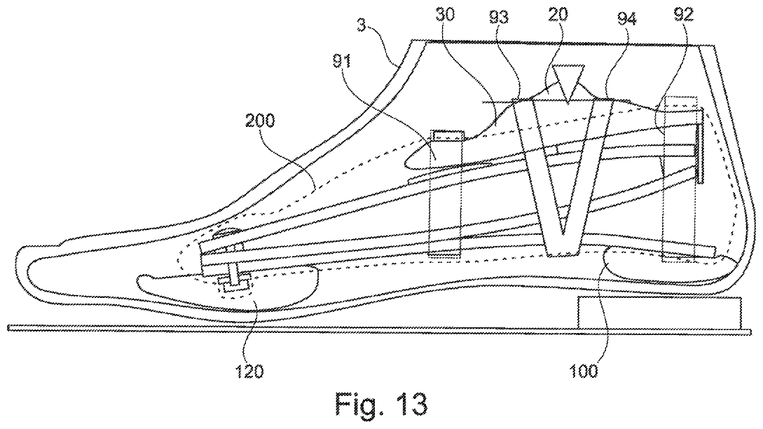

FIG. 13 shows a variant with a sheath;

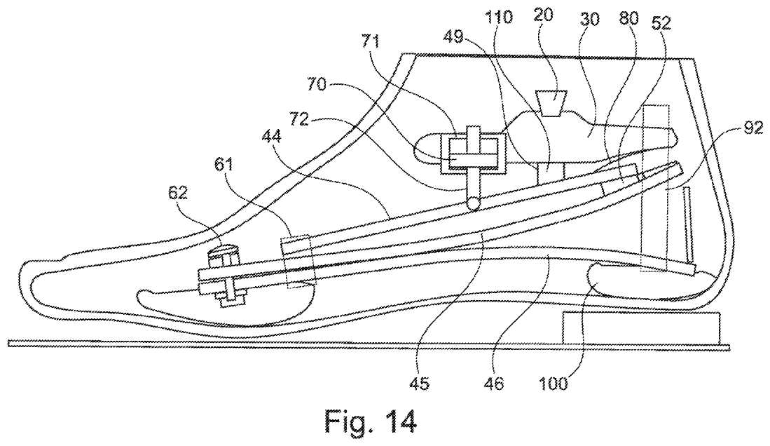

FIG. 14 shows a variant of the invention with a delay element;

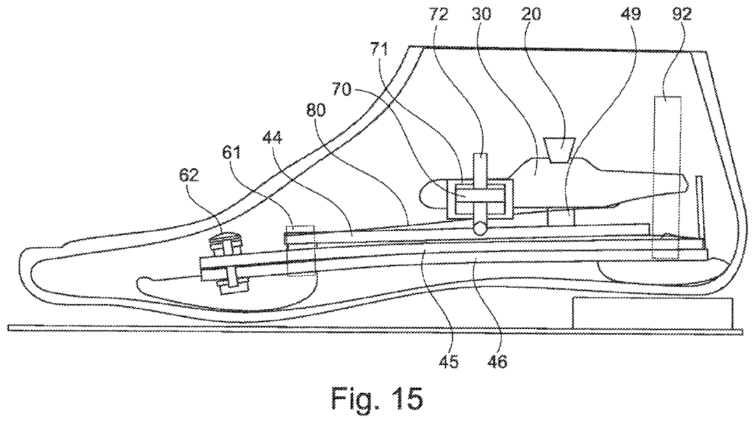

FIG. 15 shows a prosthetic foot insert according to FIG. 14 when loaded; and

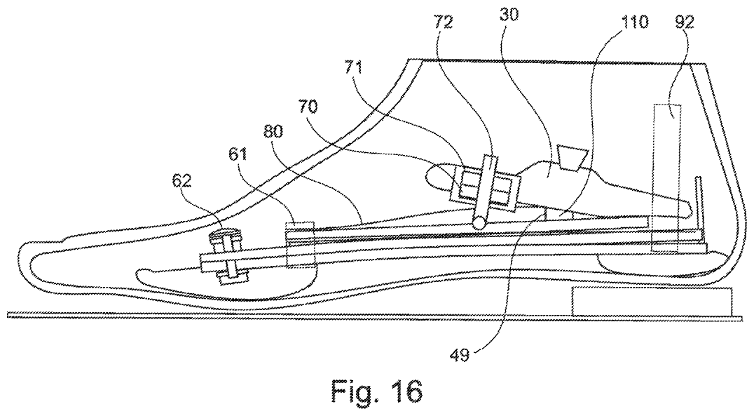

FIG. 16 shows a prosthetic foot insert according to FIG. 14 in a tilted position.

FIG. 1 shows a schematic sectional view of a prosthetic foot insert 10 with a proximal fastening device 20 in the form of a pyramid adapter, which is secured reversibly to a proximal component 2 in the form of a lower tube. Instead of a fastening device 20 in the form of a pyramid adapter, it is possible that the fastening device 20 has a receptacle for a lower-leg stump or an osseointegrated attachment to a bone. The fastening device 20 can be formed in one piece for example in the context of an additive manufacturing method and at the same time form a stump receptacle. It is likewise possible that the proximal component 2 is formed in one piece on the fastening device 20, for example likewise by an additive manufacturing method.

Distally with respect to the fastening device 20, a holder 30 is arranged which can be formed in one piece with the fastening device 20 or can be designed to be connected to a separately produced fastening device 20. The holder 30 has an adjustable receptacle 31 which protrudes posteriorly from the holder 30 and serves to receive a posterior limiting element 92. The length of the holder 31 can be fixed and set via a nut 32, which can be braced relative to the holder 30.

The holder 30 is fastened in an anterior region 41 and a posterior region 42 to a main spring 40 which, in the illustrative embodiment shown, is composed of three leaf springs 44, 45, 46. In the illustrative embodiment shown, the holder 30 is mounted on the proximal spring 44 via an anterior force introduction region 410 and a posterior force introduction region 420. Between the anterior force introduction region 410 and the holder 30, an anterior damping element 50 is arranged which lies on a receptacle 910 for an anterior limiting element 91. Between the force introduction region 410 and the proximal spring 44 below the damper 51, which can be designed as an elastomer damper for example, a pivot axis 110 is formed which extends substantially perpendicular to the drawing plane or substantially horizontally orthogonal to the sagittal plane. The holder 30 can be tilted about this pivot axis 110 relative to the main spring 40, in particular to the proximal spring 44. Both the anterior limiting element 91 and the posterior limiting element 92 are guided below the distal spring 46 or the base spring 46 and, in the illustrated unloaded state of the prosthetic foot insert 10, have a pretensioning that has been caused by the deformation of the leaf springs 44, 45, 46. All the components of the prosthetic foot insert 10 are held on one another by the pretensioning. In the region of the anterior limiting element 91, a contact element 48 is arranged between the rear leaf spring 45 or medial spring 45 and the distal spring 46, the position of which contact element 48 is displaceable in the anterior-posterior direction. The contact element 48 can be exchanged, for example in order to define the location where force is introduced between the medial spring 45 and the distal spring 46. Depending on the position in the anterior or posterior direction, the deflection behavior will change on account of different force introduction points. Moreover, a contact element 47 is arranged between the anterior end of the medial spring 45 and the anterior end of the distal spring 46, which contact element 47 is mounted displaceably or exchangeably thereon. The spring properties of the prosthetic foot insert 10 can be set via the choice of material, size and position of the contact elements 47, 48.

A guide element 80 is additionally arranged between the proximal spring 44 and the holder 30 and is secured, for example screwed, glued, welded, positively locked or clamped, to the holder 30 in the posterior region 42, i.e. in the region lying posteriorly with respect to the introduction of force into the fastening device 20. The guide element 80 extends beyond the anterior force introduction region 410 as far as a front end region of the proximal spring 44 and is designed as a spring steel sheet in order to permit tilting of the holder 30 relative to the main spring and to permit rollover of the holder 30 on the top face of the proximal spring 44 in the region of the anterior force introduction region 410.

The limiting elements 91, 92 can be designed in particular as straps or cables; they can be designed as loops and guided around the top face or bottom face of the holder or of the base spring 47. It is likewise possible that the limiting elements 91, 92, as telescopic sleeves or flexible and tensionally rigid cables, are held clamped between receptacles on the holder 30 and the base spring 47. The prosthetic foot insert 10 is embedded in a cosmetic foot cover 3 and can be exchangeably fastened to the latter.

The limiting elements 91, 92 can be designed such that they are both or individually adjustable, in particular able to be shortened or lengthened. Alternatively, a pretensioning between the one or more limiting elements 91, 92 and the holder 30 and/or the base spring 46 can be varied by inserts or spacers. Alternatively, the pretensioning can be changed by exchanging limiting elements 91, 92 of different length. In order to prevent slipping when a load is removed or when the pretensioning of the limiting element 91, 92 is canceled, the limiting element 91, 92 can be secured with form-fit engagement in the respective receptacle. For example, such a receptacle 93 with a passage or a device for securing the limiting element 92 is provided at the posterior end of the base spring 46. The receptacle 93 can at the same time assume a padding function.

If a heel load is applied that goes beyond standing, for example at heel strike, the three springs 44, 45, 46 of the base spring 40 deflect in such a way that the holder 30, via the posterior force introduction region 42, exerts an axial force in the direction of the ground and compresses the main spring 44 in the posterior region, such that the posterior limiting element 92 is relaxed, while the anterior limiting element 91 still remains tensioned. In the further course of the loading of the foot, when force is introduced substantially perpendicularly via the proximal component 2 into the fastening device 20, a uniform compression in particular of the proximal spring 44 and of the medial spring 45 takes place, such that both limiting elements 91, 92 are relaxed. If, in the course of continued walking, the proximal component is then tilted in the anterior direction, i.e. forward, the posterior end of the holder 30 lifts away from the proximal spring 44, such that a gap forms between the posterior force introduction region 420 and the proximal spring 44. The holder 30 is then secured via the guide element 80 in the medial-lateral direction and against twisting relative to the main spring 40. The rollover can take place comparatively easily without very great resistance on account of the tiltable or pivotable bearing of the holder 30 on the main spring 40. As soon as the pivoting angle is so great that the distance between the posterior end of the holder 30 and the posterior end of the base spring 46 is so great that the posterior limiting element 92 is tensioned, the base spring 46 is additionally activated and provides an additional counterforce against further pivoting toward the front. It is thereby possible to provide a serial activation of the individual springs 44, 45, 46 during a gait cycle, since the maximum pivoting travel of the holder 30 relative to the proximal spring 44 is limited. The limit is effected via the limiting element 92.

FIG. 2 shows a variant of the prosthetic foot insert 10, likewise with three springs 44, 45, 46, wherein the proximal spring 44 and the medial spring 45 are arranged relative to each other in a biconcave shape, such that an elliptical or almost elliptical free space 400 forms. In the posterior region, a free space 401 is likewise formed between the underside of the medial spring 46 and the top face of the distal spring 45, so as to permit deflection at heel strike.

The variant according to FIG. 2 has receptacles 910 and 940 in the anterior and posterior region of the holder 30, which receptacles are designed as grooves for receiving the respective limiting elements 91, 92. Receptacles 920, 930 are likewise formed on the base spring 46 and serve as a protection for the base spring 46 and at the same time prevent unwanted displacement of the respective limiting element 91, 92. The receptacle 930 at the posterior end of the base spring 46 has a through-hole, which prevents removal of the limiting element 92 even when unloading occurs. The receptacle 930 is formed in a heel pad 100; a forefoot pad 120 is formed at the anterior end of the base spring 46. Exchangeable or displaceable contact elements can be arranged between each of the springs. In the illustrative embodiment shown, these contact elements are indicated by reference sign 49 only between the posterior end of the proximal spring 44 and the medial spring 45. A damper element 52 can be arranged as elastomer damper between the holder 30 and the proximal spring 44. The anterior end of the holder 30 is connected to the spring 44 via a hinge, such that the holder 30 can pivot relative to the proximal spring 44 but cannot detach therefrom in the vertical direction. The bearing block of the hinge is connected to the proximal spring 44 with form-fit engagement in order to take up torsional forces, such that twisting about a vertical axis cannot take place. However, tilting is possible about a substantially horizontal pivot axis 110 perpendicular to the walking direction or longitudinal extent of the prosthetic foot insert. The hinge can be fastened in a posterior region 42 of the main spring 40, for example via a spring steel sheet or a spring element or, in the anterior region 41, by clamping or another form-fit connection.

FIG. 3 shows a further variant of the invention. The spring construction corresponds substantially to that of FIG. 1 or 2, but the fastening of the holder 30 to the main spring 44 via the guide element 80 is different. Whereas the guide element 80 in FIG. 1 extends from the anterior end region of the proximal spring 44 to the posterior end region of the holder 30, the illustrative embodiment shown is provided with a folded guide element 80 or a multi-part guide element 80, which is initially secured to the proximal spring 44 in the anterior region 41, for example via an elastomer damper 51 or a fastening element. From the anterior region 41, the guide element 80 extends as far as the posterior region 42 of the proximal spring 44 and from there forward again to below the anterior limiting element 91 just before the anterior damper, and it is held there on the underside of the holder 30 via a fastening element 61. By virtue of this folded design of the guide element 80, which can be designed for example as spring steel sheet, it is possible to effect forward tilting about an anterior pivot axis 110 in the region of the front force introduction region 410 and in addition to permit return tilting in the posterior region 42 about a rear pivot axis 111 above the posterior damper 52 in the posterior force introduction region 420. This rear pivot axis 111 functions for example in the event of a rearward displacement in the presence of an axial load, until the anterior limiting element 91 is made effective by a corresponding displacement and relaxation of the springs. Instead of a one-piece, folded design of the guide element, the latter can also be designed in two parts and be connected in the rear or posterior region.

FIG. 4 shows a further variant of the invention, in which the guide element 80 extends from a posterior region 42 on the main spring 40, on which it is held for example via a fastening element 62 in the form of a clip or clamp, to the anterior region 41 of the holder 30 and is either held there by clamping or secured via an anterior fastening element 61. Between the fastening elements 61, 62 and the proximal spring 44 or the holder 30, dampers 51, 52 can be arranged so as to provide a soft contact between holder 30 and spring 44 during walking. The limiting elements 91, 92 are designed as loops. The anterior loop is guided in the medial-lateral direction around the prosthetic foot insert 10; the posterior limiting element 92 is guided medially and/or laterally. It is also possible for a plurality of limiting elements 92 to be arranged medially and/or laterally on the holder 30 and the base spring 47.

FIGS. 5a to 5c show different loading phases of a prosthetic foot insert 10 in a further embodiment. FIG. 5a shows the prosthetic foot insert 10 inside the cosmetic foot cover 3 in an unloaded state or when standing relaxed with a substantially horizontally oriented holder 30, which is supported at its front end on a distal spring 46 via a double spring arrangement with a proximal spring 44 and a medial spring 45. Below the distal spring 46, a ground spring 43 is arranged on which a heel pad 100 and a forefoot pad 120 are arranged. The distal spring 46 can extend as far as the forefoot end or to the front end of the ground spring 43. A contact element 47 is arranged between the upwardly curved distal spring 46 and the base spring 43, via which contact element 47 a force introduction point can be set in particular under heel loading.

By the pretensioning of the two limiting elements 91, 92 designed as straps, the stiffness of the prosthetic foot insert 10 against tilting in the anterior-posterior direction can be set. The greater the pretensioning of the limiting elements 91, 92, the stiffer or more stable the prosthetic foot insert.

FIG. 5b is a schematic view showing the arrangement and the behavior of the prosthetic foot insert 10 under a strong heel load. The rear part of the holder 30 is loaded and presses onto the rear end of the distal spring 46, which is pressed in the direction of the posterior end of the base spring 43. Since the front end of the distal spring 46 is secured to the forefoot region of the base spring 43, the distal spring bulges in the midfoot region via the contact element 47, such that three-point bending takes place. In addition, anterior to the contact element 47, a vertically downwardly acting force is exerted via the double spring 44, 45 on account of the pretensioning by the limiting element 91. FIG. 5b indicates that the limiting element 92 is completely relaxed; posterior to the holder 30, a guide is still arranged both for the distal spring 46 and for the holder 30, which guide prevents the rear limiting element 92 from being able to slip from the holder 30.

In the case of a forefoot load, which occurs for example during rollover, the front or anterior region of the holder 30 is loaded and is supported on the distal spring 46 via the two springs 44, 45 and on the base spring 43 via the distal spring 46. The force introduction point is defined via the contact element 47 and is changed by displacement along the longitudinal extent of the springs. The proximal spring 44 and the medial spring 45 are moved toward each other, such that the free space 400 between the two springs is reduced or minimized. The rear end of the holder 30 is displaced upward, on account of the pivotable bearing on the proximal spring 44, until it comes into contact with the rear limiting element 92. In this state, the rear end of the holder 30 lifts away from the distal spring 46.

FIG. 6 shows a variant of the prosthetic foot insert 10 with a fastening element 80 in the form of a spring tongue, which is secured to the underside of the holder 30 via a screw 62 or another fastening element. The holder 30 is supported both on the proximal spring 44 and on the top of the guide element 80 via a pad 53. When the forefoot is loaded, the holder 30 tilts in the region of the pad 53 about a non-defined pivot axis, until the rear limiting element 92 prevents further pivoting relative to the proximal spring 44. The guide element 80 extends as far as the front end of the proximal spring 44 and is held there via a fastening element 81, for example a clasp, a clip or a strap. The distal spring 46 ends approximately at the same height as the proximal spring 44. The medial spring 45 extends further in the anterior direction beyond the forefoot pad 120. In the posterior region, dampers 51, 52 are arranged between holder 30 and proximal spring 44 and between proximal spring 44 and medial spring 45, in order to damp an impulse when the springs lift away from the holder 51. The dampers 51, 52 are preferably secured at one end to the holder 30 or to one of the springs 44, 45, in order to allow the components to move away from one another. In the case of highly elastic materials, the dampers 51, 52 can also be glued to the components at both ends.

FIG. 7 shows a variant of FIG. 6, having basically the same structure but, instead of a clasp or a clip as fastening element 61, having a screw which passes through all of the springs 44, 45, 46 and which prevents a displacement of the springs 44, 45, 46 along one another during loading. Pads or dampers 54, 55 are arranged between the springs 44, 45, 46. A pad element can likewise be arranged between the head of the screw 61 and the proximal spring 44 and between the nut and the distal spring 46. By the clamping in the forefoot region, the springs 44, 45, 46 are subjected to a moment during deformation, as a result of which the free spring length is shortened. The clamping by a screw 61 prevents a pushing movement and makes the overall prosthetic foot insert 10 stiffer than in a solution that allows a displacement of the springs relative to one another.

FIG. 8 shows a further variant of the invention with a guide element 80, which is designed as a hinge receptacle for the holder 30, and which can be pivoted about a pivot axis 110 about its front end. The rear end of the holder 30 is limited via the rear limiting element 92 and its maximum pivoting in respect of the distal spring 46. The guide element 80 extends into the forefoot region and is arranged on the base spring 46 via the fastening element together with the distal spring. A damper 51 is arranged between the holder 30 and the guide element 80, which extends as far as the rear end of the holder 30. A further damper 52 is likewise arranged between the guide element 80 and the rear end of the proximal spring 44.

FIG. 9 shows a variant of FIG. 8 where, instead of a base spring, a ground guide element 88 is arranged for receiving and guiding the main spring 40. The guide element 80 and the ground guide element 88 are connected to each other via the fastening element 61 in the region of the forefoot pad 120. At its front end, the guide element 80 has a lengthening tongue, via which an elastic and resilient bearing on the ground guide element 88 is realized.

FIG. 10 shows an embodiment of a variant according to FIG. 8 in which a damper 50 is arranged between the holder 30 and the fastening device 20, such that not only is the holder 30 mounted pivotably relative to the proximal spring 44 about the pivot axis 110, but the fastening device 20 is also pivotable relative to the holder 30 about the pivot axis 110. The fastening device 20 is shown in three positions: the solid line indicates the base setting, the dotted line indicates a forwardly tilted position of the fastening device 20, and the dashed line indicates a lowered and rearwardly tilted position of the fastening device 20.

Besides an embodiment as damper 50, an actuator can also be provided via which it is possible to effect a motorized adjustment as regards the inclination and therefore, for example, an adaptation to different heel heights. If the device is designed as a damper 50, a constant force or a constant moment can cause lowering or forwardly directed tilting. A slow rise or fall permits a correct setting, for example by closure of suitable control valves and by locking of the damper 50, designed as a hydraulic damper, in the desired position.

FIG. 11 shows a variant of FIG. 6 with a holder which, on the underside, has a formation that has approximately parallel to the formation on the top of the proximal spring 44. The contact element 47 between the distal spring 46 and the medial spring 45 is arranged in the region of the front limiting element 91; the pads 51, 52 in the rear region of the prosthetic foot insert are secured by the rear limiting element 92 against lateral deviation, and a guide at the rear end of the distal spring 46 prevents a rearward deviation of the pads 51, 52. The guide element 80 extends from the rear end in the posterior region 42 as far as the front end of the springs 44, 45, 46 and is secured thereon by the screw 62. The springs 44, 45, 46 and also the guide element 80 are held with clamping and form-fit engagement by the screw 62, such that, upon deformation, the springs are subjected to a thrust torque, as a result of which the free spring length overall is shortened.

In the embodiment according to FIG. 11, as in all the other embodiments, it is possible, by fixing the limiting elements 91, 92 or at least one of the limiting elements 91, 92 to the holder 30 and to the distal spring 46 or the heel pad 100, to achieve stabilization against pronation and/or supination, since it is ruled out that the respective limiting element 91, 92 slides relative to the holder 30 and/or the distal spring 46, and only a compression is allowed under a corresponding axial load.

In the illustrative embodiment according to FIG. 11, the proximal spring 44 and the medial spring 45 are shaped with an approximately parallel curvature, as a result of which the deflection space is reduced, but a gentler rollover as a whole is permitted. The distal spring 46 curves away from the medial spring 45 such that, between them, a gap or a free space forms which becomes larger in the posterior direction and which reaches approximately as far as the front limiting element 91 or the contact element 47.

FIG. 12 shows a further variant similar to the embodiment of FIG. 6, but without a guide element and with a pad 51 in the anterior region of the holder 30.

In the region below the fastening device 20, a contact element 48 is arranged between the underside of the holder 30 and the top of the proximal spring 44; the springs 44, 45, 46 are secured to one another via a screw 42 and coupled to the forefoot pad 120. The position of the pressure point or of the force introduction point is defined by the positioning of the contact element 48. As long as a heel load is present and the forefoot is not set down, the rollover point of the heel defines the force introduction point. The rear limiting element 92 is relaxed, and the springs 44, 45, 46 are compressed in the heel region and moved toward one another. The front limiting element 91 is tensioned and prevents a displacement of the holder 30 from the proximal spring 44. As soon as the forefoot is set down, the front limiting element 91, for example a strap, is relaxed, and the distal spring 46 and the rear end of the holder 30 move away from each other and tension the rear limiting element 92 up to a set maximum distance. The further the contact point between the holder 30 and the proximal spring 44 is positioned forward, i.e. in the anterior direction, the softer the axial compliance has to be set.

A further variant of the invention is shown in FIG. 13, where the spring structure corresponds substantially to that of FIG. 12. In addition to the limiting elements 91, 92, a functional sheath 200 is provided which is arranged around the spring components and the holder. The functional sheath 200 is designed like a sock and is used in addition to the cosmetic foot cover 3. The functional sheath 200 does not surround the heel pad 100 and the forefoot pad 120 in the illustrative embodiment shown. It can however also enclose these. The functional sheath 200 can be made of an inextensible high-strength material with suitable elasticity and is used in particular to reduce friction and therefore noises inside the prosthetic foot. Additional straps or tensioning elements 93, 94 can be used together with the functional sheath 200 in order to set the elastic properties and the rollover and energy transfer properties of the prosthetic foot insert 10. The tensioning elements 93, 94 can also be integrated in the functional sheath 200.

FIG. 14 shows a variant with a holder 30 at whose front end a cylinder chamber 71 for receiving a movable piston 70 is designed or arranged. The piston 70 is supported on the proximal spring 44 via a piston rod 72. In addition, the holder 30, at its posterior end, is connected to the front end of the proximal spring 44 via the guide element 80. In the front region, the guide element 80 is coupled to the spring 44 via the fastening element 61. The medial spring 45 and the distal spring 46 are connected to each other separately on each other via the screw 62.

The holder 30 is mounted on the proximal spring 44 so as to be pivotable about the pivot axis 110. At heel loading, for example at heel strike, as far as an early stance phase in which the prosthetic foot insert lies flat on the ground, the piston 70 inside the cylinder chamber 71 does not appear, such that no axial force is exerted on the proximal spring 44 via the piston rod 72. As soon as the rollover movement progresses and an increasing forefoot load is applied, the piston 70 comes into contact with the upper limit of the cylinder chamber and a compressive force is exerted on the springs via the piston rod 72. In this way, starting from a predefinable time point, an axial force is introduced into the main spring 40 and a further displacement in the forward direction or an anterior tilting is prevented or made difficult, such that an increased stability is made available to the user starting from the middle stance phase. A rollover and slight tilting about a neutral positon are permitted by the play that the piston 70 has inside the chamber 71. A restoring force can be made available by the guide element 80.

FIG. 15 shows the position in which, with a high axial load, the piston 70 abuts the top face of the chamber 71 and exerts a compressive force on the springs. The rear limiting element 92 is relaxed, and the rear end of the guide element 80 is lifted away from the top face of the proximal spring 44.

FIG. 16 shows the prosthetic foot insert according to FIGS. 14 and 15 in a state in which the holder 30 is tilted counter to the walking direction, and the piston 70 comes into contact with the underside of the cylinder chamber 71 and prevents further pivoting counter to the clockwise direction and thus further displacement of the holder 30 or of the front end of the holder 30 away from the proximal spring 44.

All the embodiments of the invention allow the prosthetic foot insert 10 to be made relatively flat, such that it is possible in principle to use the prosthetic foot insert 10 together with an additional prosthetic foot joint or to adapt it to patients with a long below-knee stump, for example an amputation stump. The small number of structural parts, which are easy to produce and do not require any complex shaping methods, facilitate production and also a robust and reliable design, which can also be easily adapted by an orthopedic technician to the different needs and customs of the respective users. The mechanical setup of the prosthetic foot inserts 10, with a small number of movable parts, requires little or no maintenance, and therefore, despite being individually adaptable and being able to be modified during the period of use, only minimal servicing has to be provided.

In most of the embodiments of the invention, three leaf springs are arranged which can be divided into two functional pairings. The proximal spring and medial spring generally work as forefoot spring, whereas the medial spring and distal spring work mainly as heel spring. The distal spring is still operative at the end of the forefoot movement. The heel, like the forefoot, is pretensioned in the unloaded state by the limiting elements, and similarly under a normal stance load. The holder is secured to the spring arrangement, generally to the proximal spring, via a guide element. By way of at least one contact element arranged between the holder and the proximal spring, the force introduction point and therefore the transition from heel load to stance phase and forefoot load are set. A second contact point in the region of the physiostable point anterior to the fastening device has the function of defining the force introduction point under forefoot loading. By the displacement of the contact points or of the contact elements between the respective springs, the spring stiffness of the overall system is changed, without the coordination of the individual springs to one another and thus the overall structure of the prosthetic foot insert being changed. Under normal loading of the prosthetic foot during standing, the axial forces are distributed uniformly to both feet of a patient. The spring pretensioning is then generally so great that the two limiting elements are still not relaxed or the posterior limiting element is still not relaxed. In other words, the pretensioning via the limiting elements or pretensioning elements is such that, during standing, the prosthetic foot insert provides sufficient stability to be able to give the user a sufficient sense of security. Upon forefoot loading, i.e. upon slight forward tilting, a gentle rollover process is initiated on account of the displaceability of the holder about a pivot axis. The energy is likewise stored in the springs, and an energy transfer is permitted from the heel spring to the forefoot load.

It is moreover possible, by the adaptation of the springs, to provide lowering in the middle stance phase, as a result of which uniform sinking and a movement adapted to the natural gait movement can be achieved. The energy stored in the springs during the middle stance phase is released upon further gait progression and facilitates the forward movement.

In particular in the landing phase of the foot after the swing phase, i.e. at heel strike, the prosthetic foot inserts provide large spring excursion in the heel. In addition, the spring pretensioning can be pretensioned via the limiting element in the heel region. Upon forefoot loading at the end of the stance phase, all three springs or all springs work together. On account of the energy transport into the forefoot during the rollover from heel strike at the end of the stance phase, the user is moved in the walking direction, since no upward vertical movement takes place during the rollover. Therefore, technically short prosthetic foot inserts are possible without the user having the feeling of falling into a hole at the end of the stance phase, since mechanically short feet give an unnatural early rollover and lower the center of gravity. With the prosthetic feet inserts, lowering in the middle stance phase is effected by the uniform spring pretensioning which, at the end of the stance phase, results in lifting when the springs in the forefoot relax.

Besides the position of the contact elements, the shape and the size of the contact elements are also crucial for the gait behavior and the energy transport. The narrower the contact elements and thus the force introduction regions, the more precise and accurate the behavior; the wider the contact elements or force introduction regions, the softer the gait sensation.

The springs are preferably oriented perpendicularly or almost perpendicularly with respect to the loading. Thus, in the case of leaf springs, the longitudinal extent is substantially perpendicular to the loading direction, as a result of which the material properties of the leaf springs are optimally utilized. The pretensioning of the springs is advantageously chosen such that, during normal standing, there is no or only minimal deformation, and therefore the force introduction point remains stable and it is possible to stand steadily without excessive stiffness.

### LIST OF REFERENCE SIGNS

2 proximal component
3 cosmetic foot cover
10 prosthetic foot insert
20 fastening device
30 holder
40 main spring
41 anterior region
42 posterior region
43 base spring
44 proximal spring
45 medial spring
46 distal spring
47 contact element
48 contact element
49 contact element
50 damper
51 damper
52 damper
53 pad
54 pad
55 pad
60 sliding bearing
61 fastening element
62 screw
70 piston
71 cylinder
72 piston rod
80 guide element
88 ground guide element
91 limiting element
92 limiting element
100 heel pad
110 pivot axis
111 pivot axis
120 forefoot pad
200 sheath
400 free space
401 free space
410 force introduction region
420 force introduction region
910 receptacle
920 receptacle
930 receptacle
940 receptacle

1. A prosthetic foot insert with
a. a proximal fastening device for securing the prosthetic foot insert to a proximal component or to a patient,
b. a holder which is arranged distally with respect to the fastening device and is connected to the fastening device, and
c. a main spring which extends into a forefoot region and is coupled to the holder, characterized in that
the holder is mounted on the main spring so as to be tiltable in the sagittal plane, wherein a posterior limiting element is arranged between the main spring and the holder and limits a displacement of the holder away from the main spring.

2. The prosthetic foot insert as claimed in claim 1, wherein the main spring is designed as a combined spring having at least one proximal spring and at least one distal spring.

3. The prosthetic foot insert as claimed in claim 2, wherein the proximal spring and the distal spring are secured to each other at points spaced apart from each other so as to form a free space.

4. The prosthetic foot insert as claimed in claim 2, wherein the proximal spring and the distal spring or the distal springs are oriented relative to each other in a biconvex shape.

5. The prosthetic foot insert as claimed in claim 1, wherein a guide element is fastened to the main spring in an anterior region or posterior region and extends in a respectively opposite direction, and the holder is mounted so as to be tiltable via the guide element.

6. The prosthetic foot insert as claimed in claim 5, wherein an anterior limiting element is arranged between the guide element and the holder or the main spring, which limiting element, in the case of a heel load, limits a displacement of an anterior end of the holder or a displacement of the main spring away from the guide element.

7. The prosthetic foot insert as claimed in claim 5, wherein the main spring and the guide element are designed as leaf springs.

8. The prosthetic foot insert as claimed in claim 1, wherein the limiting element is tensionally rigid and flexible.

9. The prosthetic foot insert as claimed in in claim 1, wherein a forefoot pad and/or a heel pad is fastened to the main spring.

10. The prosthetic foot insert as claimed in claim 1, wherein the holder is mounted on the main spring via a spring tongue, a hinge or at least one spacer element.

11. The prosthetic foot insert as claimed in claim 1, wherein the holder introduces forces into the main spring via an anterior force introduction region anterior to the fastening device and a posterior force introduction region posterior to the fastening device.

12. The prosthetic foot insert as claimed in claim 11, wherein at least one of the force introduction regions is mounted displaceably or exchangeably on the holder or the main spring.

13. The prosthetic foot insert as claimed in claim 1, wherein at least one damper device is arranged between the holder and the main spring.

14. The prosthetic foot insert as claimed in claim 1, wherein the holder is mounted on the main spring with an adjustable proximal-distal distance.

15. The prosthetic foot insert as claimed in claim 1, wherein, in the unloaded state, the main spring is elastically pretensioned with respect to the guide element by the limiting element.

16. The prosthetic foot insert as claimed in claim 1, wherein the fastening device is mounted on the holder in a displaceable and/or articulated manner.

17. The prosthetic foot insert as claimed in claim 15, wherein a damper is arranged between the fastening device and the holder.

18. The prosthetic foot insert as claimed in claim 2, wherein an exchangeable and/or displaceably mounted contact element is arranged between the proximal spring and the distal spring and/or between the distal springs.

19. The prosthetic foot insert as claimed in claim 1, wherein the holder is mounted on the main spring so as to be tiltable in the sagittal plane on a movable surface.

20. A prosthetic foot insert comprising:
a proximal fastening device for securing the prosthetic foot insert to a proximal component or to a patient;
a holder which is arranged distally with respect to the fastening device and is connected to the fastening device; and
a combined main spring having at least one proximal spring and at least one distal spring secured to each other at points spaced apart from each other so as to form a free space, the combined main spring extending into a forefoot region and is coupled to the holder;
wherein the holder is mounted on the main spring so as to be tiltable in the sagittal plane, wherein a posterior limiting element is arranged between the main spring and the holder and limits a displacement of the holder away from the main spring.
